(19) United States
(12) Patent Application Publication
(10) Pub. No.: US 2012/0304984 A1
(43) Pub. Date: Dec. 6, 2012

(54) REVERSE TRACHEAL STOMA DILATION METHOD AND APPARATUS

(75) Inventors: James Curley, Offaly (IE); Sean Morris, Roscommon (IE); Olaf Lally, Galway (IE); Alan Finneran, Tullamore (IE); Colette Breheny, Galway (IE)

(73) Assignee: Nellcor Puritan Bennett LLC, Boulder, CO (US)

(21) Appl. No.: 13/118,718

(22) Filed: May 31, 2011

(51) Int. Cl. A61M 29/02

(52) U.S. Cl. 128/200.26

(57) ABSTRACT

A reverse dilator system and method are provided, suitable for dilating a passageway into a patient airway. In one embodiment, a tracheal intubation system is provided. The tracheal intubation system includes a reverse dilator. The reverse dilator includes a shaft and a resizable portion disposed on a distal portion of the shaft. The reverse dilator is configured to dilate a tracheal passageway leading into an airway from inside the airway.

### BACKGROUND

The present disclosure relates to a tracheal dilation techniques, and more particularly to a tracheal dilation via a reverse dilation structure.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheostomy tubes, and so forth. In the latter case, the tubes are typically inserted into an opening or stoma formed in the neck and trachea of the patient. In both cases, the tubes may be used for artificial ventilation or for assisting patient ventilation. The stoma is typically formed either surgically, through a procedure such as a cricothyroidotomy, tracheostomy, or through a micro-surgical procedure such as percutaneous dilation. Cricothyroidotomy requires the use of a surgical team working in a sterilized environment to create an opening in the cricothyroid membrane, thus providing access to the patient's airway. The procedure typically involves the cauterizing of blood vessels, and typically has the patient undergoing general anesthesia.

Percutaneous dilation entails using an instrument, such as a needle or a scalpel, to make a small opening between the tracheal rings on a frontal or anterior region of the patient's neck. The needle or scalpel may then be inserted through the opening in the tracheal rings to allow a passageway into the patient's airway. A dilator, with increasing diameter from a distal tip to a proximal base, may then be pushed inwardly towards the trachea. As the dilator penetrates the stoma, the increasing diameter of the dilator may gradually expand the stoma until a desired size is reached, suitable for the insertion of the tracheostomy tube. However, the stoma may be breached to a size larger than a tracheal passageway for the tracheostomy tube, which may result in complications. Additionally, the breach may cause tears and scars in the frontal neck region.

### SUMMARY

The present disclosure provides a novel reverse dilation technique suitable for dilating, for example, a patient's stoma for the introduction of a tracheostomy tube. The reverse dilator may include an inflatable cuff or an otherwise resizable distal section having a shape useful in dilating the stoma from inside of the patient airway. That is, the reverse dilator may be inserted into the patient airway through the trachea, the resizable distal section may then be enlarged or inflated, and the dilation of the stoma may be performed beginning from an interior wall of the patient's airway rather than from an exterior neck region. Additionally, a tracheostomy tube may be inserted with the reverse dilator acting as an insertion guide for the tracheostomy tube. Indeed, the reverse dilator may include a shaft having an outside diameter (OD) sized smaller than an inside diameter (ID) of a cannula of the tracheostomy tube, useful in enabling the insertion of the reverse dilator through the cannula of the tracheostomy tube. Accordingly, the tracheostomy tube may be inserted longitudinally into the patient's airway using the outside walls of the reverse dilator as an insertion guide. The dilator cuff may then be deflated and the reverse dilator may be removed by “sliding” the dilator outwardly through the interior of the tube cannula. The tracheostomy tube may then be used to provide ventilation support. By providing for a reverse dilator and a method of reverse dilation, the stoma opening may more closely conform to the tracheostomy tube outside walls, thus minimizing any leakage through the stoma. Further, unsightly skin tears or scars caused by dilation through the frontal neck region may be minimized or eliminated.

In accordance with one embodiment, a tracheal intubation system is provided, the tracheal intubation system having a reverse dilator. The reverse dilator includes a shaft and a resizable portion disposed on a distal portion of the shaft. The reverse dilator is configured to dilate a tracheal passageway leading into an airway from inside the airway.

In a similar arrangement, a tracheal dilator may include a shaft configured to be disposed inside of a tracheostomy tube. The tracheal dilator may further include a resizable portion disposed on a distal portion of the shaft and configured to expand and contract. The tracheal dilator may additionally include a generally curved distal tip. The shaft is configured to be inserted into an airway so that the generally curved distal tip is disposed inside the airway, and the reverse dilator is configured to dilate a tracheal passageway leading into the airway from inside the airway.

Also provided is a method for dilating a trachea. The method includes creating a tracheal passageway into an airway. The method further includes inserting a reverse dilator comprising a shaft having a resizable portion into the airway through the tracheal passageway. The method additionally includes dilating the airway by pulling outwardly on the reverse dilator.

### BRIEF DESCRIPTION OF THE DRAWINGS

Advantages of the disclosed techniques may become apparent upon reading the following detailed description and upon reference to the drawings in which:

FIG. 1 is a sectional view of a patient's tracheal region and an insertion of a percutaneous needle in accordance with a prior art technique;

FIG. 2 is a sectional view of a guide wire and the percutaneous needle inserted into the tracheal region of FIG. 1;

FIG. 3 is a sectional view illustrating an embodiment of a reverse dilator disposed in a tracheal region;

FIG. 4 is a sectional view of the same arrangement of FIG. 3, illustrating an inflated dilator cuff disposed on a distal portion of the reverse dilator;

FIG. 5 is a detail sectional view of the reverse dilator of FIG. 4 abutting an interior tracheal wall taken within arc 5-5;

FIG. 6 is a detail sectional view of the reverse dilator of FIG. 5 positioned inside a tracheal airway;

FIG. 7 is a detail sectional view of a tracheostomy tube coupled to the reverse dilator of FIG. 6;

FIG. 8 is another detail sectional view of the dilator of FIG. 7 coupled to a tracheostomy tube;

FIG. 9 is a detail section view of a tracheostomy tube disposed inside an airway;

FIG. 10 is a side view of a reverse dilator including an approximately rectangular cuff;

FIG. 11 is a side view of a reverse dilator including a reverse taper cuff;

FIG. 12 is a side view of a reverse dilator including an approximately spherical cuff;

FIG. 13 is a side view of a reverse dilator having a mechanically resizable portion; and

FIG. 14 is a side view of the reverse dilator of FIG. 13 with the resizable portion in an expanded state.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

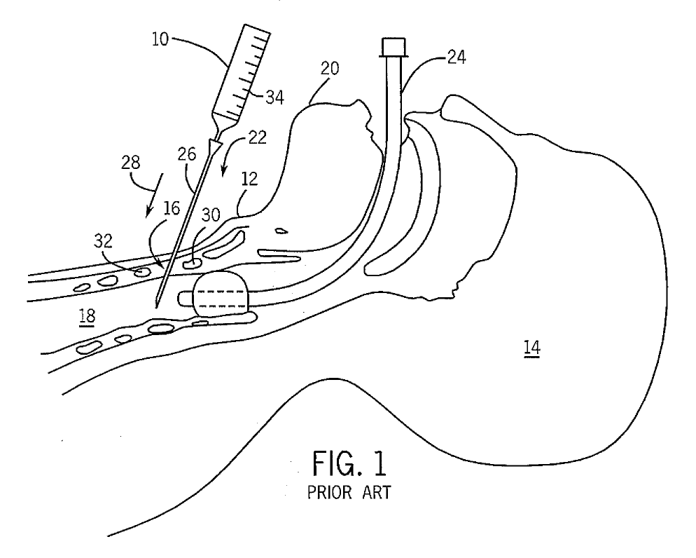

FIG. 1 is a sectional view illustrating a placement of a percutaneous needle 10 in a trachea 12 of a patient 14. By inserting the percutaneous needle 10 into the trachea 12, an initial opening or tracheal passageway 16 into an airway 18 is created, suitable for dilation. As depicted, the patient 14 is disposed in a supine position, with a chin 20 slightly elevated. In certain circumstances, a costal traction on the tracheal 12 may be applied so as to gain neck hyperextension and better access to a frontal neck region 22. General or local anesthesia may be used (e.g., 1% lidocaine solution) to dull or eliminate any discomfort during the dilation procedure. Additionally, the patient 14 may be intubated, such as by using an endotracheal tube 24. Indeed, the systems and methods disclosed herein enable a dilation procedure with artificial respiration kept in situ. It is also to be noted that the systems and methods disclosed herein enable dilation without artificial respiration support (e.g., without the endotracheal tube 24).

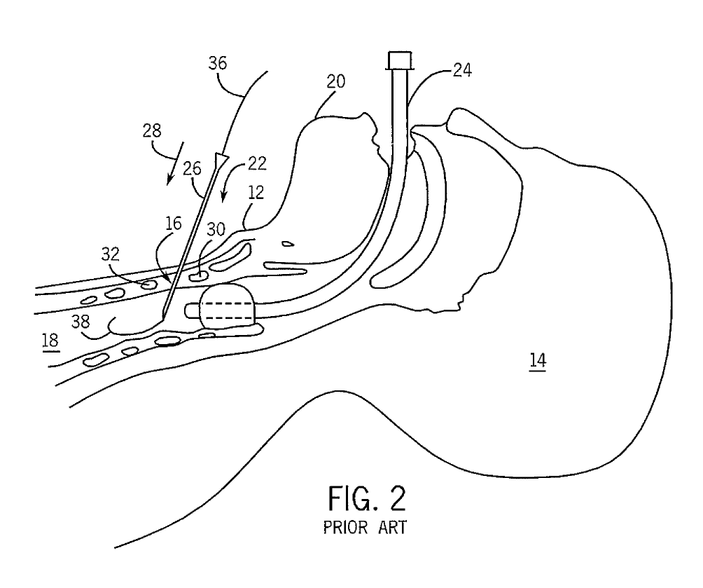

As depicted, a cannula 26 of the percutaneous needle 10 may be inserted in a direction 28, and enter the trachea 12 between a first 30 and a second 32 tracheal rings. As the percutaneous needle 10 is advanced in the direction 28, an aspiration of air through the needle 10 may indicate that the needle 26 has reached a desired position inside of the patient airway 18. Other methods useful in verifying that the cannula 26 is in the desired position may be used, such as a bronchoscopial survey, an ultrasound survey, and the like. It is also to be noted that other instruments may be used in creating the initial passageway 16 through the trachea 12, For example, a scalpel may also be used to provide a vertical or horizontal slit passageway 16 through the trachea 12, By using minimally invasive techniques to breach the trachea 12, scarring and other unsightly neck trauma may be minimized or avoided. Likewise, major bleeding during the dilation procedure may be eliminated. Once a clinician has verified that the needle cannula 26 has reached the desired position inside the airway 18, a body 34 of the needle 10 may be removed. A guide wire, such as a J-tip guide wire, may then be inserted through the cannula 26 of the needle 10, as described in more detail below with respect to FIG. 2.

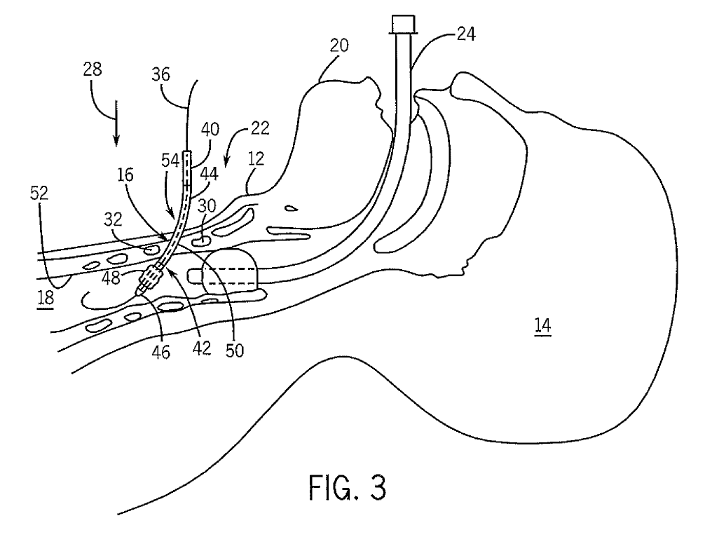

FIG. 2 is a sectional view depicting the insertion of a J-tip guide wire 36 into the patient's airway 18. Because the figure contains like elements found in FIG. 1, these elements are denoted using like reference numbers. As illustrated, the guide wire 36 is disposed inside of the needle cannula 26 and inserted so that a generally curved tip 38 is positioned inside the patient's airway 18. Using a guide wire, such as the J-tip guide wire 36, may enable a more efficient insertion of the dilation systems described herein. However, the dilation systems described herein may also be inserted into the trachea 12 without the use of any type of guide wire. When the J-tip guide wire 36 is used, the curved tip 38 may cause less trauma because the curved portion of the tip 38 is less likely to puncture the patient airway 18. That is, the curved tip 38 may prevent a “poking” or dagger effect. Once the curved tip 38 is inside the airway 18, the clinician may insert the guide wire 36 into a hollow shaft of a dilator, and then “slide” the dilator over the guide wire 36 to position a dilator partially inside of the patient airway 18, as depicted in FIG. 3.

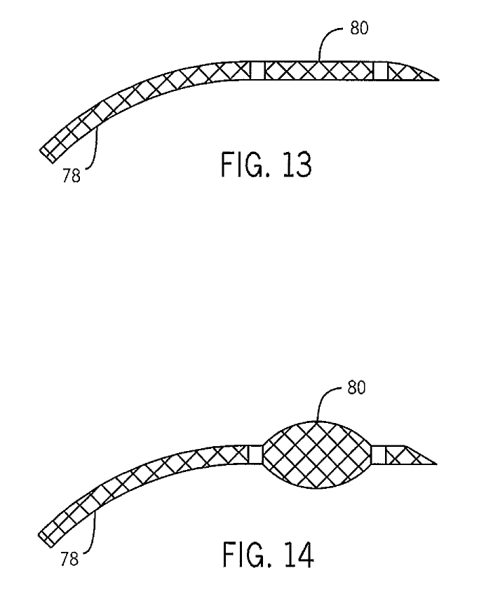

FIG. 3 is a sectional view illustrating a reverse dilator 40 having a distal portion 42 positioned inside of the patient airway 18. As mentioned above, the guide wire 36 may be disposed inside a shaft 44 of the reverse dilator 40. The reverse dilator 40 may then be “slid” in the direction 28 over the guide wire 36, thus following the contours of the guide wire 36 and entering the patient airway 18. In certain embodiments, a “punch” dilator having a diameter larger than the guide wire 36 but smaller than a diameter of the shaft 44 may be used to dilate the passageway 16 prior to the insertion of the reverse dilator 40. To aid in the insertion into the airway 18, the reverse dilator may include a generally conically-shaped distal tip 46. Additionally, the distal portion 42 of the reverse dilator 40 may include an inflatable balloon cuff 48 positioned upstream of the distal tip 46. As depicted, the cuff 48 is fully deflated during the insertion of the reverse dilator 40 to minimize an interference force between the passageway 16 and outside walls 50 of the dilator 40. It is to be noted that, in other embodiments, such as the embodiments described below with respect to FIGS. 13 and 14, the distal portion 42 of the reverse dilator 40 may include a resizable section manufactured out of shape memory alloys (e.g., Nitinol) or expandable by other mechanical techniques. Indeed, the resizable section may include either an inflatable cuff 48 or a mechanically expandable section.

Advantageously, the reverse dilator 40 may be used to dilate the tracheal passageway 16 by initiating the dilation from an interior wall 52 of the airway 18 rather than by initiating the dilation from the exterior neck region 22 of the patient 14. Indeed, the reverse dilator 40 may be inserted into the airway 18 and then “pulled” outwardly from the airway 18 through the passageway 16. In this way, the passageway 16 is dilated from inside of the airway 18. It may be beneficial to dilate through the interior wall 52 of the airway 18 because the interior wall 52 may include softer tissues offering less resistance to dilation. Further, the interior wall 52 may include natural lubrication (e.g., airway moisture) useful in reducing a reverse dilation force. Additionally, scarring on the neck region 22 of the patient may be substantially reduced because the dilation breach occurs internal to the patient. Indeed, a dilated outer diameter for a stoma 54 may be reduced.

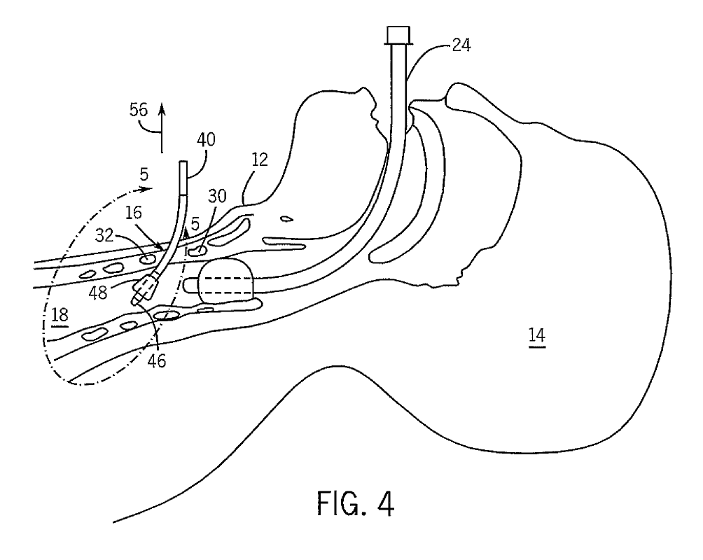

In one reverse dilation example, once the reverse dilator 40 is inserted into the desired region in the patient airway 18, the cuff 48 may then be partially or fully inflated, as depicted in FIG. 4. In this example, the partially or fully inflated cuff 48 may then be pulled outwardly through the passageway 16 in a direction 56, thus dilating the passageway 16 into a desired diameter. During emergency response procedures, it may be useful to fully inflate the cuff 40. In this way, a faster emergency response is enabled and the tracheal passageway 16 may be dilated to a diameter suitable for insertion of a variety of differently sized tracheostomy tubes. In other settings, such as an intensive care unit (ICU) setting, the clinician may select a dilator 40 including the cuff 48 of a desired diameter or inflate the cuff 48 to the desired diameter. The desired diameter is useful in accommodating a tracheostomy tube having a specific size. For example, tracheostomy tubes in a variety of sizes, such as between 2.5 to 10.5 mm ID may be dilated by inflating the cuff 48 to a desired cuff size. Other tube sizes, could, of course, be accommodated.

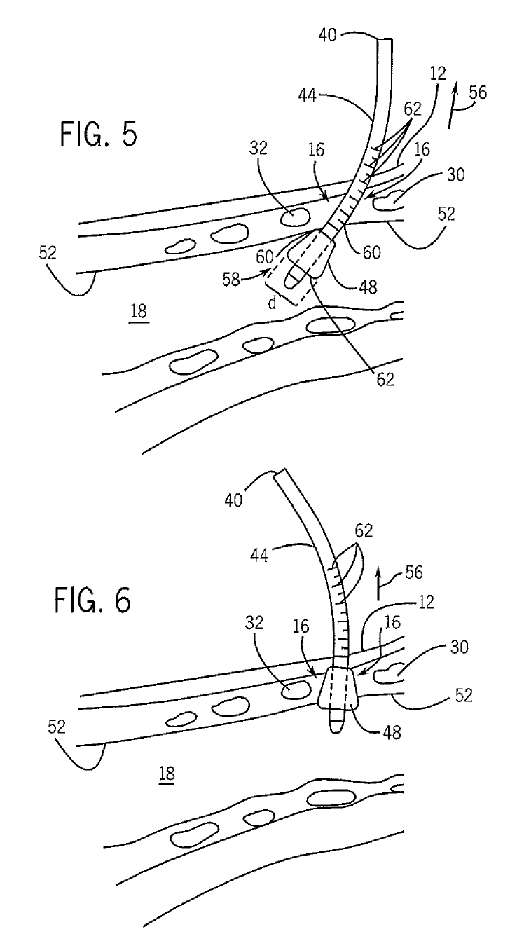

In one embodiment, a dilation cuff inflation system, such as a pump, may be used to provide a fluid flow (e.g., air flow, saline flow) to the cuff 48. The dilation cuff inflation system may use the ideal gas law, i.e., P×V=n×R×T, where P is a fluid flow pressure suitable for inflating a volume V at a temperature T based on the number of moles n of a gas and on the ideal gas constant R. Accordingly, the desired volume V for the cuff 48 may be provided by inflating the cuff 48 to the desired pressure P, taking into account temperature T, and incorporating the known values n and R, as depicted in FIG. 5. For example, the inflation P may be between about 15 cm H₂O and 100 cm H₂O. In another embodiment, the reverse dilator 40 may be manufactured in a variety of cuff 48 sizes, each cuff 48 sized to accommodate a tracheostomy tube of a given size (e.g., 2.5 to 14.5 mm OD). In this embodiment, the cuff 48 may be fully inflated so as to expand to its manufactured size.

FIG. 5 is a detailed sectional view illustrating an embodiment of the reverse dilator 40 of FIG. 4 with the cuff 48 inflated to a desired diameter d. As mentioned above, the desired diameter d may be derived by using the ideal gas law or the cuff 48 may be manufactured to be fully inflated to the diameter d. Further, the diameter d is generally derived to accommodate a tracheostomy tube having an OD approximately equal to d. Accordingly, the passageway 16 may be dilated to a size suitable for enabling the entry of a tracheostomy tube into the airway 18, while minimizing tissue trauma and scarring resulting from the dilation procedure. Indeed, the reverse dilator 40 may include other features, such as a generally conical shape 58 of the cuff 48, useful in minimizing the dilation effort and in lessening tissue trauma.

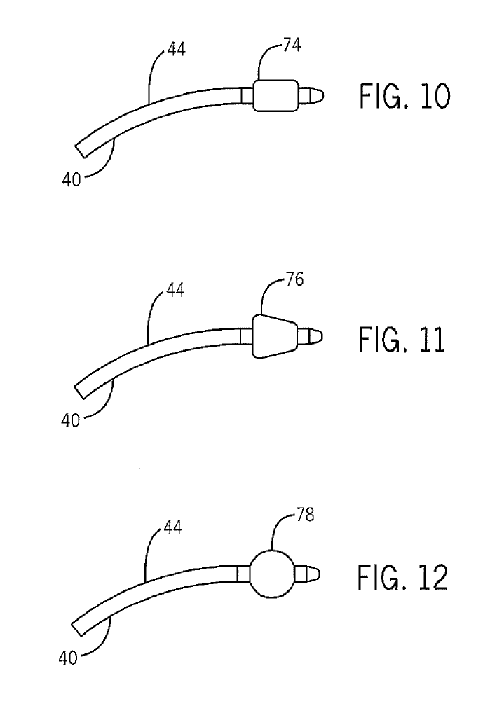

As depicted, the conical shape 58 increases in diameter, starting with a first diameter approximately equal to a diameter of the shaft 44 at a cuff attachment point 60 and ending in the diameter d at the base 62 of the cuff 48. As the reverse dilator 40 is pulled outwardly from the airway 18, the cuff attachment point 60 first makes contact with the interior wall 52 of the airway 18. By having a smaller diameter attachment point 60 as part of the cone shape 58, the cuff 48 may enable a smoother entry and dilation of the passageway 16 backwards through the interior wall 52 of the airway 18. Additionally, the cuff 48 may securely circumferentially encircle and “hug” the attachment point 60 to reduce trauma and insertion force. That is, the cuff 48 mating at the attachment point 60 may allow a smoother insertion through the interior wall 52 by eliminating protrusions or grooves at the attachment point 60. It is to be noted that other cuff shapes may be used, such as the cuff shapes described in more detail below with respect to FIGS. 10-12. It is also to be noted that, in other examples, the cuff 48 may be first fully deflated when penetrating into the interior wall 52. That is, the clinician may pull the reverse dilator 40 outwardly to position the fully deflated cuff 48 partially or fully in the passageway 16. The position of the cuff may be visually tracked by using markings 62 disposed on the shaft 44. Once the deflated cuff 48 is positioned and tracked by using the markings 62, the cuff 48 may then be inflated. The inflation of the cuff 48 inside of the passageway 16 may thus dilate the passageway 16. By inflating the fully deflated cuff 48 once the cuff 48 is inside the passageway 16, less pulling force may be used to position the reverse dilator 40 inside of the passageway 16.

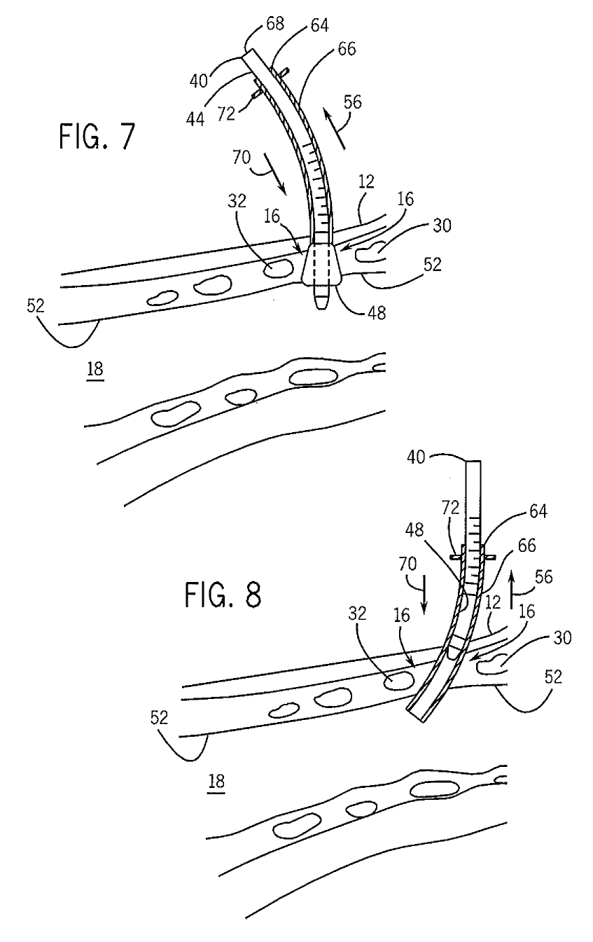

FIG. 6 is a sectional view of an embodiment of the cuff 48 of the reverse dilator 40 disposed inside the passageway 16. In the depicted example, the cuff 48 may have been positioned in the passageway 16 either inflated (partially or fully), or fully deflated. If positioned fully deflated, the clinician may then have inflated the cuff 48. As mentioned above, the markings 62 may visually aid the clinician in tracking the position of the cuff 48 with respect to the passageway 16 to a desired position. In the illustrated position, the passageway 16 is now dilated at a size suitable for enabling the insertion of a tracheostomy tube, as depicted in FIG. 7.

FIG. 7 is a sectional view on an embodiment of a tracheostomy tube 64 coupled to the reverse dilator 40. More specifically, the reverse dilator 40 has been disposed inside a cannula 66 of the tracheostomy tube 64. Indeed, an ID of the cannula 66 of the tracheostomy tube is approximately the same size as an OD of the shaft 44 of the reverse dilator 40. In this way, the reverse dilator 40 may be used as an insertion guide into the airway 18. For example, the clinician may insert a proximal end 68 of the dilator 40 into the cannula 66 and “slide” the tracheostomy tube 64 in a direction 70, following the outer walls of the shaft 44. Alternatively, the clinician may completely remove the dilator 40 by grasping the proximal end 68 and then pulling outwardly in the direction 56. The clinician may then insert the tracheostomy tube 64 into the dilated passageway 16. By providing for a guide into the airway 18, the reverse dilator 40 may aid the clinician in more efficiently disposing the tracheostomy tube 64 at a desired position, as described in more detail with respect to FIG. 8 below.

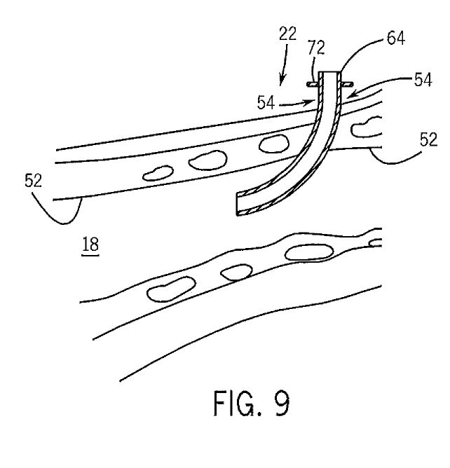

FIG. 8 is a sectional view of the reverse dilator 40 used as a guide to position the tracheostomy tube 64 into the airway 18. As mentioned above, the reverse dilator 40 may be inserted into the cannula 66 of the tracheostomy tube 64 and used to guide the tracheostomy tube 64 into the patient airway 18. In the depicted example, the cuff 48 of the reverse dilator 40 has been fully deflated to reduce or eliminate an interference fit or friction between the reverse dilator 40 and the tracheostomy tube 64. The tracheostomy tube 64 may then be pushed inwardly in the direction 70 towards the passageway 16. Likewise, the reverse dilator 40 may then be pulled outwardly in the direction 56 away from the airway 18. For example, the tracheostomy tube 64 may be pushed inwardly until a set of flanges 72 approach approximately near the trachea 12. The reverse dilator 40 may then be fully removed from the cannula 66 of the tracheostomy tube 64, as depicted in FIG. 9.

FIG. 9 depicts the tracheostomy tube 64 fully inserted into the airway 18 and ready to be used for respiratory support. Further, the reverse dilator 40, as shown in FIG. 8, has been removed to enable a connection of the tracheostomy tube 64 to, for example, a ventilator. By dilating the tracheal passageway from the inside the interior walls 52, unsightly tears or scars to the neck region 22 may be reduced. Likewise, trauma to patient tissue may also be reduced. Additionally, the diameter of the stoma 54 may more conformably fit the OD of the tracheostomy tube 64.

FIGS. 10-12 are side views illustrating various types of inflatable cuffs that may be included with the reverse dilator 40. For example, FIG. 10 illustrates a rectangular cuff 74 disposed on the distal portion of the reverse dilator 40. The rectangular cuff 74 may be useful in achieving a symmetrical dilation of the tracheal passageway 16 shown in the preceding figures. By using the rectangular cuff 74, the passageway 16 may experience equal dilation forces when compared to using asymmetrical cuffs. Of course, it is to be understood that asymmetrical cuffs may also be used, such as cuffs asymmetrical about the shaft 44 of the reverse dilator 40. The symmetrical dilation forces resulting from the use of the rectangular cuff 74 may apply equal pressure to tissues in the passageway 16 and surrounding areas, thus resulting in equal trauma, if any. In certain embodiments, the cuff 74 may be manufactured out of a resilient material, such as polyurethane, latex, rubber, vinyl, a soft polyvinylchloride, a thermoplastic elastomer (e.g., polyether block amide or PEBAX™), a silicone, and the like. The shaft 44 may be manufactured out of a more rigid material, such as polyvinylchloride, polyurethane, thermoplastic elastomers, a polycarbonate plastic, silicon, ABS, or a polyvinyl chloride (PVC). All of the cuffs and shafts described with respect to all the figures herein may use like materials.

FIG. 11 illustrates a reverse tapered cuff 76 disposed on the distal portion of the reverse dilator 40. The reverse cuff 76 may be useful in applying more pressure near the proximal end of the tracheal passageway 16, e.g., closer to the stoma 54 shown in FIG. 9. For example, it may be beneficial to re-open a stoma that had been previously used in tracheostomy and then allowed to close. Accordingly, the reverse cuff 76 may be used by first positioning the reverse dilator 40 with the reverse cuff 76 fully deflated inside of the tracheal passageway 16, and then fully inflating the reverse cuff 76. In this way, the stoma may be more efficiently re-opened.

FIG. 12 Illustrates a generally spherical cuff 78 included in the reverse dilator 40. The spherical cuff 78 may be more useful in situations where the tracheal passageway 16 may include nearby regions benefiting from higher dilation pressures. For example, should the tracheal passageway 16 be located substantially close to a tracheal ring (e.g., rings 30 or 32), slightly higher pressures may be useful in displacing the ring outwardly from the passageway 16 so as to provide an adequate dilation of the passageway 16. Accordingly, the spherical cuff 78 may be used, which may provide higher displacement pressures and a higher displacement height as compared to the cuffs 74 and 76 shown in FIGS. 10 and 11, respectively. It is to be understood that other cuff shapes may be used with the reverse dilator 40, such as a square shape, an oval shape, and so forth.

In another reverse dilator embodiment, such as the embodiment depicted with reference to FIG. 13, the dilation may be accomplished mechanically rather than through an inflatable cuff For example, in one embodiment, a reverse dilator 78 may be manufactured out of a shape memory alloy, such as equiatomic nickel-titanium (e.g., Nitinol) useful in expanding a resizable section 80. Other alloys may include copper-aluminum-nickel, silver-cadmium, cobalt-nickel-aluminum, and the like. Shape memory alloys are alloys which “remember” their original, cold-forged shaped. Accordingly, the alloys may return to a preformed shape by applying heat, such as the heat provided by the patient's tissues. In another embodiment, the reverse dilator 78 may include a spring-driven expandable mechanism, a screw-driven expandable mechanism, or any other mechanical device useful in mechanically expanding the resizable section 80.

In the shape memory alloy embodiment, FIG. 13 depicts the reverse dilator 78 in a “rested” or unheated state. In this state, the reverse dilator 78 may exhibit a geometry useful in smoothly inserting the reverse dilator 78 into the tracheal passageway 16. Once the resizable section 80 of the reverse dilator 78 is inside the airway 18 (or inside the tracheal passageway 16), the dilator 78 may begin to expand due to the body temperature, as illustrated in FIG. 14. Indeed, the resizable section 80 of the reverse dilator 78 may expand due to the memory effect associated with shape memory allows. In the spring-driven or screw-driven embodiments, FIG. 13 depicts the reverse dilator in a contracted state. Applying the spring-driven or screw-driven mechanism may then expand the resizable section 80, as shown in FIG. 14.

It is to be noted that the portion 80 may be manufactured to take on a variety of shapes, including the cuff shapes described above with respect to FIGS. 10-12. For example, the portion 80 may be cold-forged out of a Nitinol wire mesh to take on a rectangular, square, reverse tapered, spherical, or oval shape. The reverse dilator 78 may thus be used in a generally similar manner as the dilator 40 depicted in the previous figures.

What is claimed is:

1. A tracheal intubation system comprising:
a reverse dilator having a shaft and a resizable portion disposed on a distal portion of the shaft, wherein the reverse dilator is configured to dilate a tracheal passageway leading into an airway from inside the airway.

2. The system of claim 1, wherein the resizable portion comprises an inflatable cuff.

3. The system of claim 1, wherein the inflatable cuff comprises a conical shape.

4. The system of claim 1, wherein the inflatable cuff comprises a rectangular shape or a square shape.

5. The system of claim 1, wherein the inflatable cuff comprises a spherical shape or an oval shape.

6. The system of claim 3, wherein the conical shape comprises a first diameter at a distal base of the inflatable cuff and a second diameter at a proximal attachment point of the inflatable cuff to the shaft, and the first diameter is greater than the second diameter.

7. The system of claim 3, wherein the conical shape comprises a first diameter at a distal base of the inflatable cuff and a second diameter at a proximal attachment point of the inflatable cuff to the shaft, and the first diameter is smaller than the second diameter.

8. The system of claim 1, wherein the shaft comprises a plurality of markings configured to provide a visual representation of a position of the resizable portion with respect to the airway.

9. The system of claim 1, wherein the resizable portion comprises a shape memory alloy, a spring-driven mechanism, a screw-driven mechanism, or a combination thereof.

10. The system of claim 1, comprising a tracheostomy tube having tracheostomy tube cannula comprising an inner diameter (ID) approximately equal to or larger than an outer diameter (OD) of the shaft, wherein the reverse dilator is configured to be disposed inside the tracheostomy tube cannula.

11. A tracheal dilator comprising:
a shaft configured to be disposed inside of a tracheostomy tube;
a resizable portion disposed on a distal portion of the shaft and configured to expand and contract; and
a generally curved distal tip, wherein the shaft is configured to be inserted into an airway so that the generally curved distal tip is disposed inside the airway and the reverse dilator is configured to dilate a tracheal passageway leading into the airway from inside the airway.

12. The tracheal dilator of claim 11, wherein the shaft has an inner diameter (ID) approximately equal to an outer diameter (OD) of a tracheostomy tube cannula.

13. The tracheal dilator of claim 11, wherein the generally curved distal tip comprises a conical section.

14. The tracheal dilator of claim 11, wherein the resizable portion comprises an inflatable cuff

15. The tracheal dilator of claim 11, wherein the resizable portion comprises a shape memory alloy, a spring-driven mechanism, a screw-driven mechanism, or a combination thereof.

16. A method for dilating a trachea comprising:
creating a tracheal passageway into an airway;
inserting a reverse dilator comprising a shaft having a resizable portion into the airway through the tracheal passageway;
dilating the tracheal passageway by pulling outwardly on the reverse dilator,

17. The method of claim 16, wherein the dilating the tracheal passageway by pulling outwardly on the reverse dilator comprises expanding the resizable portion inside the airway and then pulling outwardly to position the resizable portion in an interior of the tracheal passageway.

18. The method of claim 16, wherein the dilating the tracheal passageway by pulling outwardly on the reverse dilator comprises pulling outwardly to position the resizable portion in an interior of the tracheal passageway and then expanding the resizable portion inside the interior of the tracheal passageway.

19. The method of claim 16, wherein the resizable portion comprises an inflatable cuff.

20. The method of claim 16, wherein the resizable portion comprises a shape memory alloy, a spring-driven mechanism, a screw-driven mechanism, or a combination thereof.
